(19) United States
(12) Patent Application Publication
(10) Pub. No.: US 2026/0060613 A1
(43) Pub. Date: Mar. 5, 2026

(54) DEHYDRATION NOTIFICATION SYSTEM

(71) Applicant: SKINOS CO., LTD., Ueda-shi, Nagano (JP)

(72) Inventors: Hideya MOMOSE, Matsumoto-shi, Nagano (JP); Mieko TAKASAKA, Matsumoto-shi, Nagano (JP)

(73) Assignee: SKINOS CO., LTD., Ueda-shi, Nagano (JP)

(21) Appl. No.: 19/106,741

(22) PCT Filed: Sep. 11, 2023
(86) PCT No.: PCT/JP2023/032959

§ 371 Date: Feb. 26, 2025

(30) Foreign Application Priority Data
Sep. 16, 2022 (JP) 2022-148273

(51) Int. Cl. A61B 5/00; A61B 5/0205; A61B 5/0537

(52) U.S. Cl. CPC A61B 5/486; A61B 5/02055; A61B 5/0537; A61B 5/4266; A61B 5/4875; A61B 5/7275; A61B 2560/0247

(57) ABSTRACT

A dehydration notification system includes: one or more types of measuring units that perform one or more types of measurement selected from a group including water loss amount variation factor measurement, water loss amount measurement, body influence measurement, body water amount measurement, supplied water amount measurement, body feature measurement, and lifestyle measurement; and an informing unit that makes notifications being two or more types of notifications that are derived from two or more types of measurement data selected from a group including water loss amount variation factor measurement data, water loss amount measurement data, body influence measurement data, body water amount measurement data, supplied water amount measurement data, body feature measurement data, and lifestyle measurement data, which are obtained from the measurement, and being notifications related to a dehydration symptom.

### TECHNICAL FIELD

The present invention relates to a dehydration notification system that makes a notification in order to avoid an occurrence of a dehydration symptom and/or alleviate the dehydration symptom.

### BACKGROUND ART

The dehydration symptom occurs due to a decrease in the water amount (hereinafter, referred to as a body water amount) held in the body. The larger reduction rate (hereinafter, moisture reduction rate) of the moisture that occupies the body weight results in the severer dehydration symptom. Recognized are respective symptoms of “thirst” at the moisture reduction rate of 2%, “strong thirst, haze, anorexia” at the moisture reduction rate of 3%, “skin flushing, irritation, body temperature rise, exhaustion, decrease and concentration of urine volume” at the moisture reduction rate of 4%, “headache, feeling of sweltering heat” at the moisture reduction rate of 5%, “body shaking, convulsion” at the moisture reduction rate of 8 to 10%, and “anuria, death” at the moisture reduction rate of 20% or more.

In regard to techniques of determining a dehydration state, disclosed is a technique of performing preliminary measurement of a subject during normal state time, and determining a dehydration state by using blood flow data obtained by the preliminary measurement and blood flow data in each measurement (PTL 1).

Moreover, in regard to techniques of determining a degree of risk of thermal injury including a dehydration symptom, disclosed is a technique of acquiring data on a heart rate, data on a body temperature, and data on a skin blood flow of a living body via a wearable sensor terminal, and determining a degree of risk of thermal injury including a dehydration symptom by comparing the acquired data with teacher data in which a plurality of sets of data on a heart rate, a deep body temperature, and a skin blood flow measured in advance with respect to a living body to be tested are classified for each risk degree level (PTL 2).

Both of the water amount (hereinafter, water intake amount) that enters the body and the water amount (hereinafter, water loss amount) that exits to the outside of the body vary largely among individuals and within the same individual (hereinafter, intraindividual variation), and in particular, it is known that the water loss amount varies largely from hour to hour even in the same individual due to various factors such as an activity state, a physical condition, a season, and a surrounding environment. In particular, when the water loss amount increases during exercise and due to perspiration, diarrhea, vomiting, polyuria, bleeding, and the like to lose the balance of the water intake amount, the body water amount decreases, the dehydration symptom easily becomes severe, and an attention is required.

The body water amount decreases with age, and is 70 to 80% of the body weight for children, approximately 60% of the body weight for adults, and approximately 50% of the body weight for elderly persons. Moreover, it is known that a gender and a lifestyle also relate to the body water amount. In elderly persons and the like originally having a small body water amount, the dehydration symptom easily becomes severe, and a special attention is required.

In both of PTLs 1 and 2, a determination is made using vital data on a subject during normal state time as reference data, and a decrease in the determination accuracy due to differences among individuals can be avoided, but no intraindividual variation is considered.

Moreover, in both of PTLs 1 and 2, the presence or absence of a risk of a dehydration symptom during measurement is merely determined by a comparison between vital data during measurement and vital data during normal state time.

The dehydration symptom has various occurrence processes, for example, the dehydration symptom may occur in a state where the perspiration amount is large or the dehydration symptom may occur in a state where the perspiration amount is small. Although the water supply is necessary as measures against the dehydration symptom, in a case where a risk of the dehydration symptom increases in a state where the perspiration amount is large, an occurrence of the dehydration symptom can be more effectively avoided by prompting the perspiration to be suppressed, in addition to the water supply.

However, as described above, in both of PTLs 1 and 2, the presence or absence of a risk of a dehydration symptom during measurement is merely determined, but it is impossible to prompt an action in which the occurrence processes are considered.

As mentioned above, in the related arts of PTLs 1 and 2, and the like, in regard to an action for avoiding an occurrence of a dehydration symptom and/or alleviating a symptom of the dehydration symptom, it is impossible to prompt a more suitable action by considering an intraindividual variation and/or an occurrence process.

### CITATION LIST

### Patent Literature

PTL 1: JP 2017-196474 A
PTL 2: JP 2018-19821 A

### SUMMARY OF INVENTION

### Technical Problem

The present invention is made in view of such circumstances, and aims to provide a dehydration notification system related to an action for avoiding an occurrence of a dehydration symptom and/or alleviating a symptom of the dehydration symptom, and capable of prompting a suitable action by considering an intraindividual variation and/or an occurrence process.

In the present invention, “dehydration symptom” includes a state where a bodily fluid amount decreases because a balance between the water amount that enters a body and the water amount that exits to the outside of the body is lost but there is no subjective symptom, and “for avoiding an occurrence of a dehydration symptom” also includes “for performance maintenance”.

### Solution to Problem

The present invention provides the following [1] to [20].

[1]

A dehydration notification system including:
one or more types of measuring units that perform one or more types of measurement selected from a group including water loss amount variation factor measurement, water loss amount measurement, body influence measurement, body water amount measurement, supplied water amount measurement, body feature measurement, and lifestyle measurement; and
an informing unit that makes notifications being two or more types of notifications that are derived from two or more types of measurement data selected from a group including water loss amount variation factor measurement data, water loss amount measurement data, body influence measurement data, body water amount measurement data, supplied water amount measurement data, body feature measurement data, and lifestyle measurement data, which are obtained from the measurement, and being notifications related to a dehydration symptom.
   [2]

The dehydration notification system according to [1], in which the notification is selected from a group including a notification related to an action for avoiding an occurrence of a dehydration symptom and/or alleviating a symptom of the dehydration symptom, a notification related to a degree of risk that a dehydration symptom occurs, and a notification related to severity of a dehydration symptom.

[3]

The dehydration notification system according to [1] or [2], further including a determining unit that derives the notification from the measurement data.

[4]

The dehydration notification system according to [3], in which the determining unit derives one type of a notification from two or more types of measurement data.

[5]

The dehydration notification system according to [3] or [4] including two or more types of the determining units, in which at least two types of the determining units respectively derive notifications different from each other from one type of common measurement data.

[6]

The dehydration notification system according to any of [3] to [5], in which the determining unit includes one or more types of functions selected from a group including A, B, and C below.
Function A) a function that converts primary data being measurement data of the measuring unit into secondary data serving as an evaluation index of a dehydration symptom
Function B) a function that detects a sign of a dehydration symptom from a determination reference stored in advance, and the primary data and/or the secondary data
Function C) a function that determines a method of the notification based on the primary data and/or the secondary data
   [7]

The dehydration notification system according to [6], in which the method determined in the C) includes one or more types selected from a group including notification timing, a notification method, a notification item, and notification intensity.

[8]

The dehydration notification system according to [6] or [7], in which a determination reference of the B) is in that measurement data to be acquired from a biosensor that acquires one or more types of measurement information selected from a group including an activity amount, a heart rate, a blood pressure, an oxygen intake amount, a respiratory rate, a deep body temperature, a skin temperature, a skin impedance, a perspiration amount, and a blood flow rate remains high.

[9]

The dehydration notification system according to any of [6] to [8], further including a holding unit that stores the measurement data, in which the determination reference is derived using the measurement data stored in the holding unit.

[10]

The dehydration notification system according to any of [1] to [9], in which the measuring unit is any one selected from a group including (A) to (D) below.
(A) a perspiration sensor including one or more types of sensors that measure an amount of body water to be generated from a skin surface, and one or more types of evaporation promotion mechanisms that prompt evaporation
(B) a perspiration sensor including two or more types of sensors that measure an amount of body water to be generated from a skin surface
(C) a perspiration sensor including one or more types of sensors that measure an amount of body water to be generated from a skin surface, a moisture-proof mechanism, and a discharge unit of a liquid
(D) a perspiration sensor including: a housing capsule including an opening portion to be attached to a skin surface, the housing capsule including an air suction hole for sucking natural air into an inside of the housing capsule, a mixture chamber that communicates with the opening portion to diffuse sweat on the skin surface, and in which the diffused sweat and the natural air are mixed to become mixed air, and an air discharge hole for discharging the mixed air from the mixture chamber; a first humidity sensor for measuring humidity of the natural air; and a second humidity sensor for measuring humidity of the mixed air
   [11]

The dehydration notification system according to any of [1] to [10], in which
the measuring unit is a biosensor to be used by being worn to a body, and
the biosensor acquires one or more measurement information selected from a group including an activity amount of the body, a heart rate, a pulse wave, a blood pressure, an oxygen intake amount, a respiratory rate, a deep body temperature, a skin temperature, a skin impedance, a perspiration amount, and a blood flow rate.
   [12]

The dehydration notification system according to any of [1] to [11], in which the measuring unit is a unit that measures a content rate of blood hemoglobin and/or a unit that measures an NaCl concentration in sweat.

[13]

The dehydration notification system according to any of [1] to [12], in which the measuring unit is a unit that measures one or more types selected from a group including a blood vasopressin concentration, a urine specific gravity, a urine osmotic pressure, and a urine volume.

[14]

The dehydration notification system according to any of [1] to [13], in which the measuring unit is a moisture measuring unit that uses a bioelectric impedance analysis method and/or a unit that measures a hematocrit value.

[15]

The dehydration notification system according to any of [1] to [14], in which the measuring unit acquires eating-and-drinking information including an intake amount and a frequency of food and drink having a diuretic effect and/or exercise information including an exercise habit and an exercise experience.

[16]

The dehydration notification system according to any of [1] to [15], in which the measuring unit acquires one or more types of information selected from a group including a water intake amount, an intake habit of food and drink having a diuretic effect, an exercise habit, and an exercise experience.

[17]

The dehydration notification system according to any of [1] to [16], in which the measuring unit acquires supplied water amount information.

[18]

The dehydration notification system according to any of [1] to [17], in which the measuring unit acquires information including one or more types selected from a group including a gender, an age, a BMI, and a body composition.

[19]

The dehydration notification system according to any of [1] to [18], in which the measuring unit acquires information including one or more types selected from a group including a season, a temperature, a humidity, and a heat index (WBGT).

[20]

A dehydration notification system including:
a measuring unit that performs two or more types of measurement selected from a group including water loss amount variation factor measurement, water loss amount measurement, body influence measurement, body water amount measurement, supplied water amount measurement, body feature measurement, and lifestyle measurement; and
an informing unit that makes notifications being two or more types of notifications that are derived from two or more types of measurement data selected from a group including water loss amount variation factor measurement data, water loss amount measurement data, body influence measurement data, body water amount measurement data, supplied water amount measurement data, body feature measurement data, and lifestyle measurement data, which are obtained from the two or more types of the measurement, and being notifications related to a dehydration symptom.

### Advantageous Effects of Invention

With the present invention, it is possible to provide a dehydration notification system related to an action for avoiding an occurrence of a dehydration symptom and/or alleviating a symptom of the dehydration symptom, and capable of prompting a suitable action by considering an intraindividual variation and/or an occurrence process.

### BRIEF DESCRIPTION OF DRAWINGS

FIG. 1 is a diagram explaining a configuration of a dehydration notification system in a first embodiment.

FIG. 2 is a diagram explaining a system configuration in a modification example of the first embodiment.

FIG. 3 is a diagram explaining a system configuration in another modification example of the first embodiment.

FIG. 4 is a diagram explaining a configuration of a dehydration notification system in a second embodiment.

FIG. 5 is a diagram explaining FIG. 4 as a specific example.

FIG. 6 is a diagram explaining a system configuration in a modification example of the second embodiment.

FIG. 7 is a diagram explaining FIG. 6 as a specific example.

FIG. 8 is a diagram explaining a system configuration in another modification example of the second embodiment.

FIG. 9 is a diagram explaining FIG. 8 as a specific example.

FIG. 10 is a diagram explaining a system configuration in a still another modification example of the second embodiment.

FIG. 11 is a diagram explaining FIG. 10 as a specific example.

FIG. 12 is a diagram explaining a configuration of a dehydration notification system in a third embodiment.

FIG. 13 is a diagram explaining the third embodiment as a specific example.

FIG. 14 is a diagram explaining the third embodiment as a specific example including an apparatus configuration.

### DESCRIPTION OF EMBODIMENTS

Hereinafter, a dehydration notification system in first to fourth embodiments of the present invention will be described in details. Further, the present invention is not limited to the following embodiments.

### First Embodiment_Measuring Unit+Informing Unit

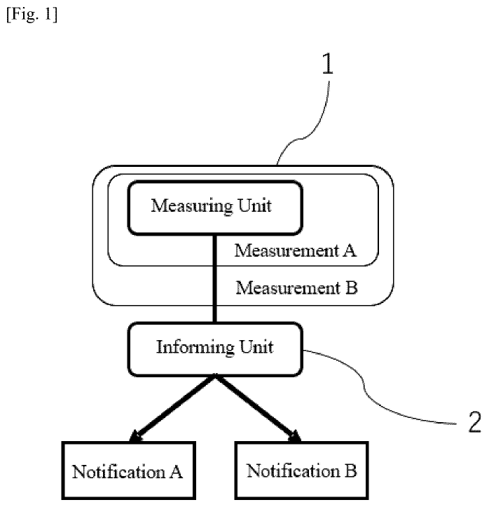

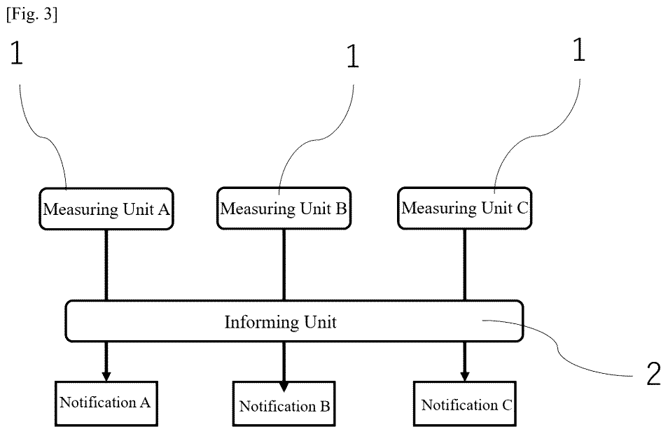

A dehydration notification system according to one embodiment of the present invention includes, as illustrated in FIGS. 1 to 3, one or more types of measuring units 1 that perform one or more types of measurement selected from a group including water loss amount variation factor measurement, water loss amount measurement, body influence measurement, body water amount measurement, supplied water amount measurement, body feature measurement, and lifestyle measurement, and an informing unit 2 that makes notifications being two or more types of notifications that are derived from two or more types of measurement data selected from a group including water loss amount variation factor measurement data, water loss amount measurement data, body influence measurement data, body water amount measurement data, supplied water amount measurement data, body feature measurement data, and lifestyle measurement data, which are obtained from the one or more types of the measurement, and being notifications related to a dehydration symptom.

### (Notification)

The notification related to a dehydration symptom may preferably be a notification selected from a group including making a notification of an action for avoiding an occurrence of a dehydration symptom and/or alleviating a symptom of the dehydration symptom (hereinafter, referred to as an avoidance and/or alleviation action notification), making a notification of a degree of risk that a dehydration symptom occurs in the future (hereinafter, referred to as a risk notification), and making a notification of severity of a dehydration symptom that occurs at the current time point or in the future (hereinafter, referred to as a severity notification).

A combination of two or more types of the notifications is not specially limited, but preferably includes the avoidance and/or alleviation action notification.

Examples of the avoidance and/or alleviation action notification include a notification of prompting immediate water supply, a notification of prompting movement to a cool place to take a rest, a notification of having a body feature that easily causes the dehydration symptom, and a notification of prompting an increase in a daily water intake amount.

In the present description, examples of the body feature, which is a body feature that has an influence on the water loss amount and the body water amount, include an age, a gender, a BMI, a body composition, and a birthplace.

An example of the risk notification includes a notification of an increase in the perspiration amount.

An example of the severity notification includes a notification of a larger body influence due to an increase in the moisture loss by the perspiration.

In the present description, the body influence indicates a parameter related to a body change that deviates from a control range of an adjustment system of a body, and examples of the parameter include a heart rate, a deep body temperature, AVP, renin, NaCl, uric acid, and urea nitrogen.

Making two or more types of notifications that are derived from two or more types of measurement data can more effectively avoid the occurrence of a dehydration symptom, compared with the technique of merely determining the presence or absence of a risk of a dehydration symptom during measurement by a comparison between vital data during measurement and vital data during normal state time.

### (Measurement Data)

Measurement data that derives the notification is a combination of two or more types selected from a group including water loss amount variation factor measurement data, water loss amount measurement data, body influence measurement data, body water amount measurement data, supplied water amount measurement data, body feature measurement data, and lifestyle measurement data.

### (Measuring Unit)

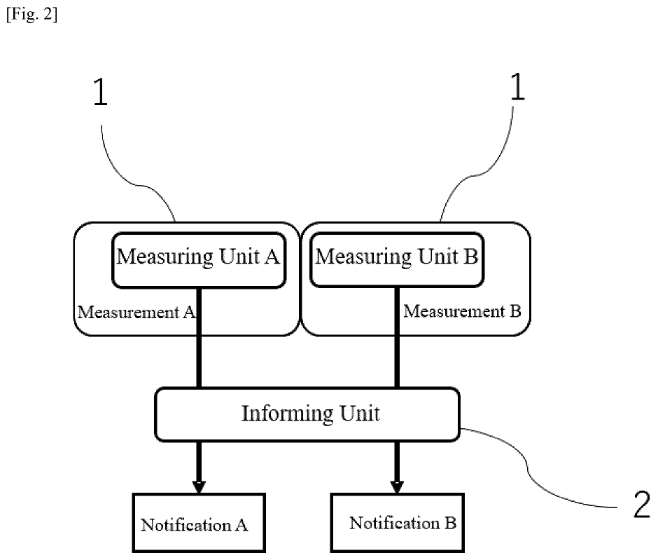

One type of a measuring unit may obtain two types of measurement data as illustrated in FIG. 1, or two types of measuring units may obtain two types of measurement data as illustrated in FIG. 2.

A preferable measuring unit for each measurement will be described below.

### (Measurement 1_Water Loss Amount Variation Factor Measurement)

A water loss amount varies depending on the perspiration amount and the urine volume, and the variation results from factors such as the exercise intensity and the intake of food and drink having a diuretic effect.

The water loss amount variation factor measurement indicates measurement of a parameter that becomes a variation factor of the water loss amount.

Examples of the parameter include an activity amount, a heart rate, a blood pressure, an oxygen intake amount, a respiratory rate, a deep body temperature, a skin temperature, a skin impedance, a perspiration amount, a blood flow rate, and an intake amount of food and drink having a diuretic effect.

The measuring unit of a water loss amount variation factor is a biosensor to be used by being worn to a body, and is preferably a biosensor that acquires one or more types of measurement information selected from a group including an activity amount of the body, a heart rate, a blood pressure, an oxygen intake amount, a respiratory rate, a deep body temperature, a skin temperature, a skin impedance, a perspiration amount, and a blood flow rate.

With the biosensor, it is possible to obtain biosensor measurement data related to at least one type selected from a group including an activity amount of the body, a heart rate, a blood pressure, an oxygen intake amount, a respiratory rate, a deep body temperature, a skin temperature, a skin impedance, a perspiration amount, and a blood flow rate. Secondary data indicating an influence on the perspiration amount can be derived from the biosensor measurement data serving as primary data.

Examples of preferable measuring units for the measurement of the respective parameters include an acceleration sensor as an activity amount measuring unit, a heartbeat sensor as a heart rate measuring unit, an oscillometric method and a Rivaloch Korotkoff method as a blood pressure measuring unit, an expiration gas analysis as an oxygen intake amount measuring unit, a breath sensor as a respiratory rate measuring unit, a temperature sensor as a skin temperature measuring unit, a deep body thermometer as a deep body temperature measuring unit, a ventilation capsule type perspiration meter as a perspiration amount measuring unit, an impedance meter as a skin impedance measuring unit, a laser Doppler blood flow meter as a blood flow rate unit, and an intake information input tool, such as a questionnaire related to food and drink having a diuretic effect, as an intake amount measuring unit of an intake amount of food and drink having a diuretic effect.

The exercise intensity that has an influence on the perspiration amount can be measured by using the acceleration sensor, the heartbeat sensor, the oscillometric method or the Rivaloch Korotkoff method, the expiration gas analysis, the breath sensor, the temperature sensor, the deep body thermometer, the ventilation capsule type perspiration meter, and the laser Doppler blood flow meter. The appearance of the perspiration amount can be qualitatively checked by using the impedance meter.

The study by the present inventors has confirmed that a phenomenon “remaining high” in which a value of measurement data by one or more types selected from a group including the acceleration sensor, the heartbeat sensor, the oscillometric method or the Rivaloch Korotkoff method, the expiration gas analysis, the breath sensor, the temperature sensor, the deep body thermometer, the ventilation capsule type perspiration meter, the laser Doppler blood flow meter, and the impedance meter remains in a high state is generated at a substantial initial stage of a dehydration symptom, so that the dehydration symptom can be detected based on the data remaining high at a premonitory stage before the dehydration symptom exhibits a serious symptom.

An example of a notification that is derived from the water loss amount variation factor measurement data includes an avoidance and/or alleviation action notification, specifically, a notification of prompting earlier water supply for performance maintenance. It is considered that a decrease in performance starts when the moisture reduction rate is about 1% during sports, and the notification can be made by detecting a possibility of a decrease in the body water amount from the activity amount data and intake amount data on food and drink having a diuretic effect.

### (Measurement 2_Water Loss Amount Measurement)

The measurement of a water loss amount indicates measurement of a parameter related to the amount of water exiting to the outside of the body.

Examples of the parameter include a perspiration amount, a urine volume, and an expiration body water amount (≠respiratory rate).

The measuring unit of a water loss amount is preferably any perspiration sensor selected from a group including (A) to (D) below.
(A) a perspiration sensor including one or more types of sensors that measure an amount of body water to be generated from a skin surface, and one or more types of evaporation promotion mechanisms that prompt evaporation
(B) a perspiration sensor including two or more types of sensors that measure an amount of body water to be generated from a skin surface
(C) a perspiration sensor including one or more types of sensors that measure an amount of body water to be generated from a skin surface, a moisture-proof mechanism, and a discharge unit of a liquid
(D) a perspiration sensor including: a housing capsule including an opening portion to be attached to a skin surface, the housing capsule including an air suction hole for sucking natural air into an inside of the housing capsule, a mixture chamber that communicates with the opening portion to diffuse sweat on the skin surface, and in which the diffused sweat and the natural air are mixed to become mixed air, and an air discharge hole for discharging the mixed air from the mixture chamber; a first humidity sensor for measuring humidity of the natural air; and a second humidity sensor for measuring humidity of the mixed air

The measuring unit of a water loss amount is preferably an environment information input tool that inputs information related to one or more types selected from a group including a season, a temperature, a humidity, and a heat index (WBGT).

Examples of another measuring unit include a weight scale, a filter paper method, a measurement cup for urine volume measurement, and an absolute humidity sensor.

Perspiration amount measurement data can be obtained by the perspiration sensor.

Environmental data such as a season, a temperature, a humidity, and a heat index (WBGT) can be obtained by the environment information input tool. Secondary data indicating an influence on the perspiration amount and the urine volume can be derived from the environment data serving as primary data.

Perspiration amount measurement data can be obtained by the weight scale and the filter paper method.

Urine volume measurement data can be obtained by the measurement cup for urine volume measurement.

Expiration body water amount measurement data can be obtained by the absolute humidity sensor.

Examples of a notification that is derived from a result of the measurement of the water loss amount include an avoidance and/or alleviation action notification and a risk notification, specifically, a notification of prompting earlier water supply for performance maintenance and a notification of an increase in the perspiration amount.

### (Measurement 3_Body Influence Measurement)

The measurement of a body influence indicates measurement of a parameter related to a body change that deviates from a control range of the adjustment system of the body.

Examples of the parameter include a heart rate, a deep body temperature, vasopressin (AVP), renin, NaCl, uric acid, and urea nitrogen.

The measuring unit of a body influence is a biosensor to be used by being worn to a body, and is preferably a biosensor that acquires one or more types of measurement information selected from a group including a heart rate, a pulse wave, a blood pressure, a respiratory rate, a deep body temperature, a skin temperature, a skin impedance, a skin temperature, and a blood flow rate.

Examples of another measuring unit includes an immunoassay method, conductivity, ICP, a potentiometric titration method, a Mohr method, ion chromatography, a Contaminometer, capillary electrophoresis, HPLC, a reduction method, an enzyme method, and a urease GLDH-UV method.

One or more types of biosensor measurement data selected from a group including a heart rate, a pulse wave, a blood pressure, a respiratory rate, a deep body temperature, a skin temperature, a skin impedance, a skin temperature, and a blood flow rate can be obtained by the biosensor. Secondary data indicating an influence on the body can be derived from the biosensor measurement data serving as primary data. Examples of secondary data indicating an influence on the body can include secondary data indicating an influence on the circulatory system due to the blood concentration, and secondary data indicating whether to appropriately perform heat dissipation.

Examples of a preferable measuring unit for measurement of each parameter include a heartbeat sensor as a heart rate measuring unit, a photoplethysmographic sensor as a pulse wave measuring unit, an oscillometric method and a Rivaloch Korotkoff method as a blood pressure measuring unit, a breath sensor (generally, changes in a pressure, a flow rate, a temperature, a humidity, and the like) as a respiratory rate measuring unit, a deep body thermometer as a deep body temperature measuring unit, an impedance meter as a skin impedance measuring unit, a temperature sensor as a skin temperature measuring unit, a laser Doppler blood flow meter as a blood flow rate measuring unit, an immunoassay method as an AVP measuring unit, an immunoassay method as a renin measuring unit, conductivity, ICP, a potentiometric titration method, a Mohr method, an ion chromatography, a Contaminometer, or capillary electrophoresis as an NaCl measuring unit, PLC, a reduction method, or an enzyme method as an uric acid measuring unit, a urease GLDH-UV method as an urea nitrogen measuring unit.

The heart rate increases due to the blood concentration, so that secondary data indicating an influence on the circulatory system due to the blood concentration can be derived from the heart rate measurement data serving as primary data.

The pulse wave indicates an expansion change of a blood vessel in a certain site as a waveform, and the blood becomes difficult to flow due to the blood concentration to provide an influence on a waveform of the pulse wave, so that secondary data estimating the extent of the blood concentration can be derived from the pulse wave measurement data serving as primary data.

When the blood becomes difficult to flow due to the blood concentration, the blood pressure increases, so that secondary data estimating the extent of the blood concentration can be derived from the blood pressure measurement data serving as primary data.

Secondary data estimating an influence on the circulatory system can be derived from the respiratory rate measurement data serving as primary data.

Secondary data determining whether the heat dissipation is appropriately performed can be derived from the deep body temperature measurement data serving as primary data.

Secondary data related to the electrolyte of sweat can be derived from the skin impedance measurement data serving as primary data.

Secondary data estimating an increase in the deep body temperature from the skin temperature can be derived from the skin temperature measurement data serving as primary data.

The blood flow rate indicates the amount of blood flowing in a certain site as a waveform, and the blood becomes difficult to flow due to the blood concentration to provide an influence on the waveform of the blood flow rate, so that secondary data estimating an extent of the blood concentration can be derived from the blood flow rate measurement data serving as primary data.

AVP measurement data reflects the amount of AVP secretion of which is accelerated due to an increase in the plasma osmotic pressure, a decrease in the circulation plasma amount, and a reduction in the blood pressure. Secondary data indicating the progression of dehydration can be derived from the AVP data serving as primary data.

Renin measurement data reflects the amount of renin that is secreted by sensing the blood pressure. Secondary data indicating the progression of dehydration can be derived from the renin measurement data serving as primary data.

NaCl measurement data reflects a state of the resorption delay of NaCl. Secondary data indicating the progression of dehydration can be derived from the NaCl measurement data serving as primary data.

Uric acid measurement data reflects a state of the uric acid resorption overexpression due to a decrease in the urine volume and an effect by the antidiuretic hormone. Secondary data indicating the progression of dehydration can be derived from the uric acid measurement data serving as primary data.

Urea nitrogen measurement data reflects a state of the urea nitrogen resorption overexpression due to a decrease in the urine flow rate and an effect by the antidiuretic hormone. Secondary data indicating the progression of dehydration can be derived from the urea nitrogen measurement data serving as primary data.

Examples of the notification that is derived from the body influence measurement data include a severity notification and an avoidance and/or alleviation action notification, specifically, a notification of a larger body influence due to an increase in the moisture loss by the perspiration and a notification of prompting the cancellation of work with the perspiration.

### (Measurement 4_Body Water Amount Measurement)

The body water amount indicates measurement of a parameter that reflects the amount of water held in the body.

Examples of the parameter include a body water amount, a content rate of blood hematocrit (Ht), a content rate of blood hemoglobin (Hb), a urine specific gravity, a urine osmotic pressure, a urine volume, total protein, and albumin.

Examples of a measuring unit of the body water amount include a bioelectric impedance method (BIA method), a microhematocrit method, an automatic blood cell counter, immunochromatography, an SLS-Hb method, a Triton/NaOH method, an absorbance method, a weight method, a hydrometer, a refractometer method, a test paper method, a freezing point depression method, a static pressure method, a dynamic pressure method, a compensation method, a BCA method, a Bradford method, a Lowry method, and a modified BCP method, and a BCG method.

Body water amount measurement data can be obtained by the bioelectric impedance method (BIA method).

Ht measurement data can be obtained by the microhematocrit method, the automatic blood cell counter, and the immunochromatography. The Ht value, which becomes relatively high as the moisture in the body decreases, can be used as an index of insufficient moisture.

Hb measurement data can be obtained by the SLS-Hb method, the Triton/NaOH method, and the absorbance method. The Hb value, which becomes relatively high as the moisture in the body decreases, can be used as an index of insufficient moisture.

Urine specific gravity measurement data can be obtained by the weight method, the hydrometer, the refractometer method, and the test paper method. The urine specific gravity, which becomes relatively high as the moisture in the body decreases, can be used as an index of insufficient moisture.

Urine osmotic pressure constant data can be obtained by the freezing point depression method, the static pressure method, the dynamic pressure method, and the compensation method. The urine osmotic pressure, which becomes relatively high as the moisture in the body decreases, can be used as an index of insufficient moisture.

Total protein measurement data can be obtained by the BCA method, the Bradford method, and the Lowry method. The total protein, which becomes relatively high as the moisture in the body decreases, is used as an index of insufficient moisture.

Albumin measurement data can be obtained by the modified BCP method and the BCG method. Albumin, which becomes relatively high as the moisture in the body decreases, can be used as an index of insufficient moisture.

Examples of the notification that is derived from the body water amount measurement data includes a risk notification and an avoidance and/or alleviation action notification, specifically, a notification of a tendency that the body water amount is chronically small, a notification of prompting a habit of water replacement to be made, and a notification of prompting exercise, work and the like with the perspiration to be prevented.

### (Measurement 5_Supplied Water Amount Measurement)

The supplied water amount measurement indicates measurement of a parameter that reflects the supplied water amount.

An example of a supplied water amount measuring unit can include an information input tool that inputs information related to the supplied water amount.

Supplied water amount measurement data can be obtained by the supplied water amount measuring unit.

An example of the notification that is derived from the supplied water amount measurement data includes an avoidance and/or alleviation action notification, specifically, a notification of a tendency that the body water amount is chronically small, and a notification of prompting a habit of water replacement to be made.

### (Measurement 6_Body Feature Measurement)

The body feature measurement indicates a parameter related to a body feature that provides an influence on the water loss amount and the body water amount.

Examples of the parameter include an age, a gender, a BMI, and a body composition.

BMI measurement data and body composition constant data serve as body features that provide an influence on the body water amount because muscle contains the moisture overwhelmingly more than fat, and thus the body water amount of the cycloid type tends to be less than that of the leptosomatic type.

Examples of a preferable measuring unit for each parameter include an information input tool for a questionnaire and the like related to an age, a gender, and the like, a height and weight scale as a BMI measuring unit, and a bioelectric impedance method (BIA method) as a body composition measuring unit.

Secondary data indicating an influence on dehydration due to the body feature can be derived from the data serving as primary data obtained by the information input tool.

An Example of the notification derived from the body feature measurement data includes a risk notification, specifically, a notification of a tendency that the body water amount is small and the dehydration symptom easily occurs.

### (Measurement 7_Lifestyle Measurement)

The lifestyle measurement indicates measurement of a parameter related to a daily routine and a past experience.

An example of a lifestyle measuring unit can include an information input tool that inputs information related to a water intake amount as a daily habit, an intake habit of food and drink having a diuretic effect, an exercise habit, an exercise experience, and the like.

Secondary data indicating an influence on the dehydration due to the lifestyle can be derived from the data serving as primary data obtained by the information input tool.

An Example of the notification derived from the lifestyle measurement data includes a risk notification, specifically, a notification of a tendency that the body water amount is small and the dehydration symptom easily occurs.

### Second Embodiment_Measuring Unit+Informing Unit+Determining Unit

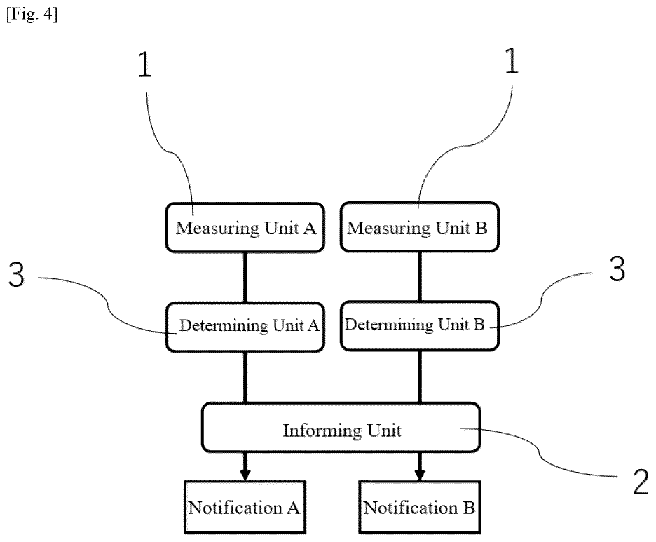

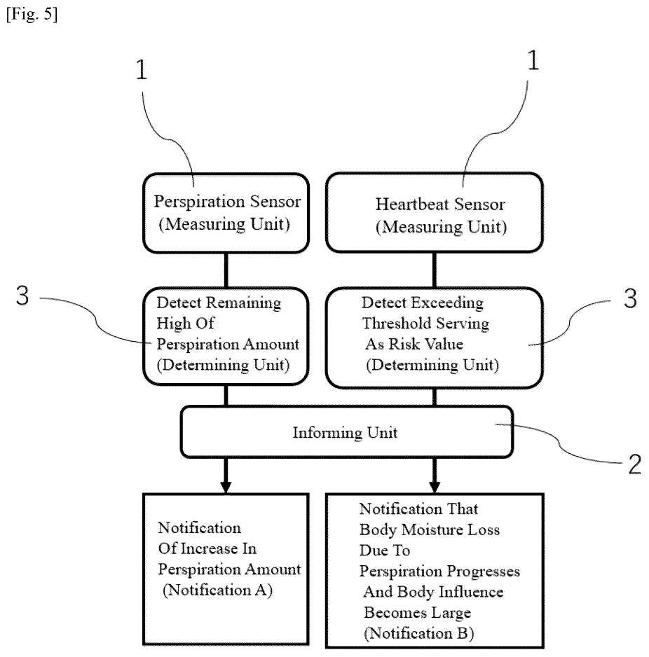

A dehydration notification system in one embodiment of the present invention includes, in addition to the first embodiment, as illustrated in FIGS. 4 to 11, a determining unit 3 that derives the notification from the measurement data.

### (Determining Unit 1)

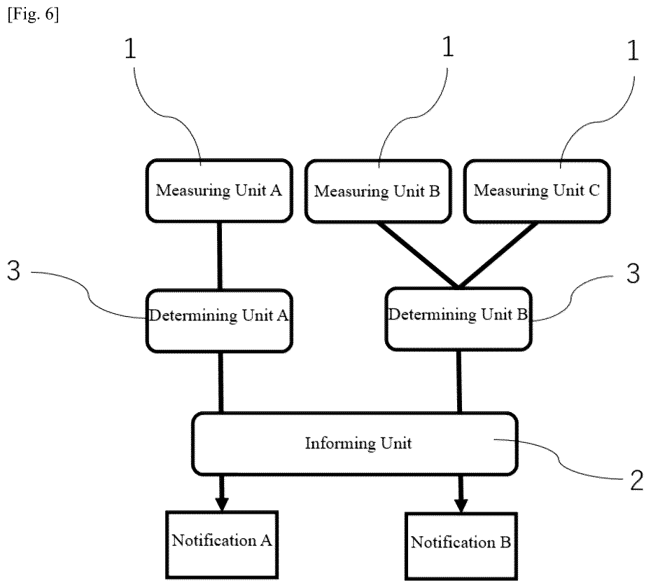

The determining unit may be, as in FIG. 6, a determining unit A that derives one type of a notification from one type of measurement data obtained from one type of a measuring unit, or may be a measuring unit B that derives one type of a notification from two types of measurement data obtained from two types of measuring units.

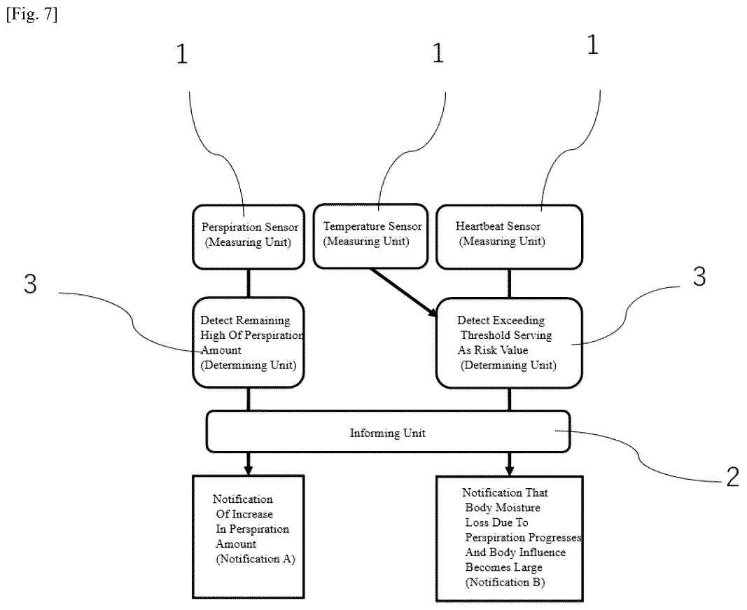

A specific example in which the measuring unit that derives one type of a notification from two types of measurement data obtained from two types of measuring units is used includes, as illustrated in FIG. 7, an example in which temperature data measured by a temperature sensor and heartbeat data measured by a heartbeat sensor are used, the determining unit makes a determination by lowering a threshold serving as a risk value in terms of heartbeat data because it is assumed that a body moisture loss due to the perspiration is likely to progress in a case where the temperature is high, and the determining unit makes a notification that the body moisture loss due to the perspiration progresses and a body influence becomes large when having detected the heartbeat data exceeding the threshold serving as the risk value. Two types of measurement data are used to make a determination in this manner, so that it is possible to prompt a suitable action.

### (Determining Unit 2)

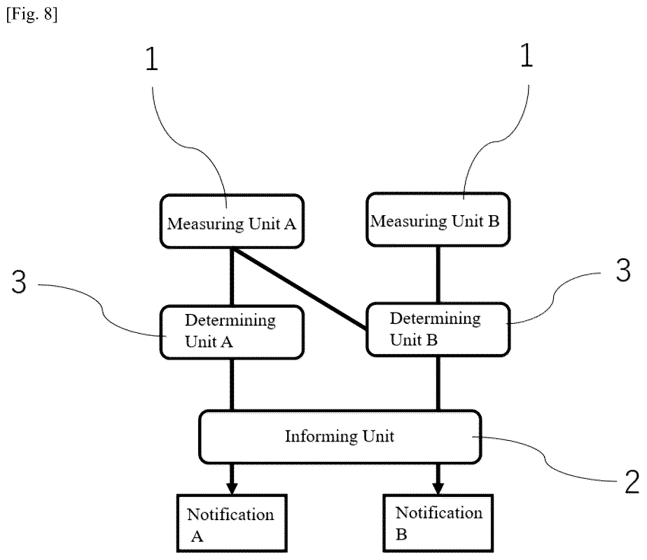

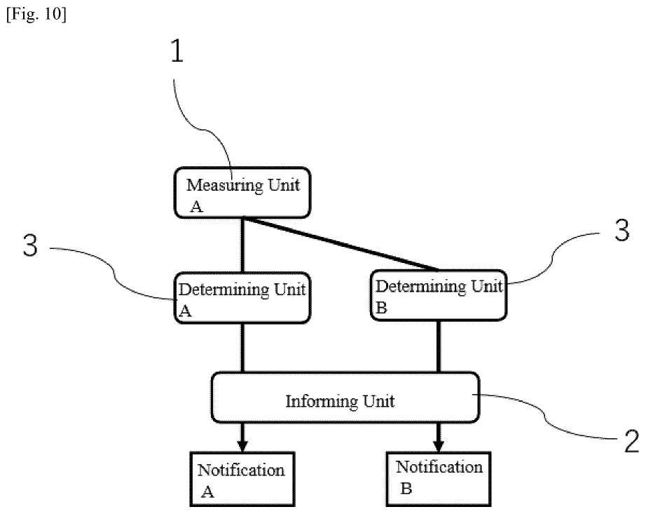

A determining unit may derive notifications different from each other from one type of measurement data obtained from one type of a measuring unit, as in the determining units A and B in FIGS. 8 and 10.

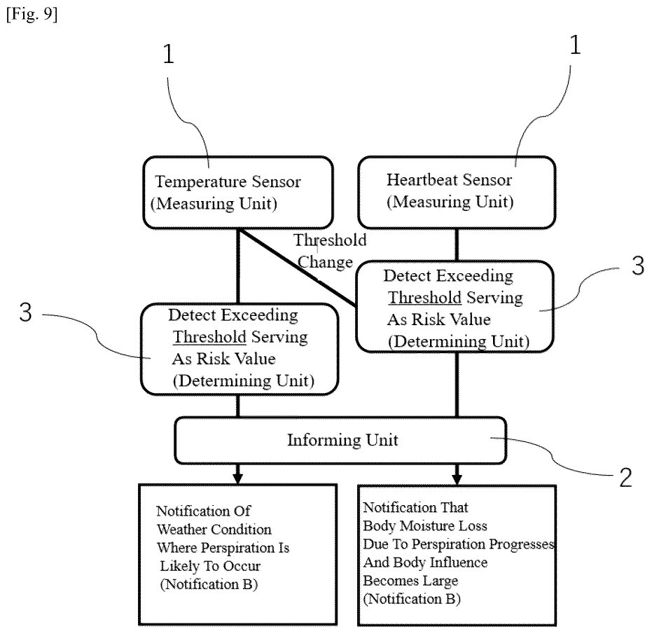

A specific example in which two determining units derive notifications different from each other from one type of measurement data obtained from one type of a measuring unit includes, as illustrated in FIG. 9, an example in which temperature data measured by a temperature sensor is used, the determining unit A detects the temperature data exceeding a threshold serving as a risk value and makes a notification of a weather condition where the perspiration is likely to occur, and the determining unit B detects the temperature data exceeding a threshold serving as a risk value and makes a notification that a body moisture loss due to the perspiration progresses and a body influence becomes large.

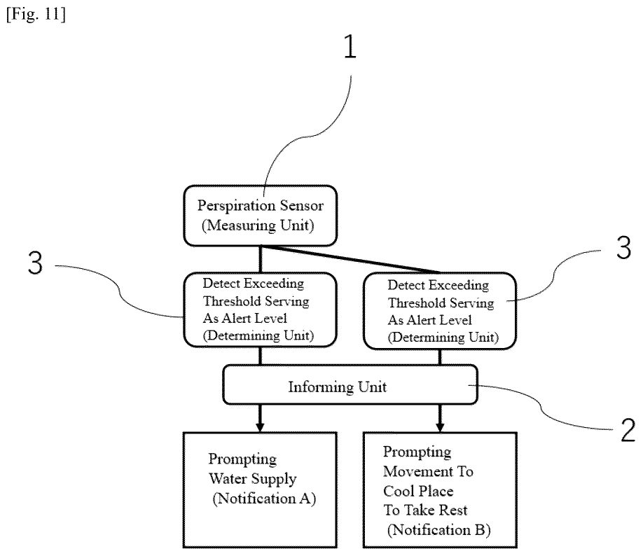

Another specific example includes, as illustrated in FIG. 11, an example in which perspiration data measured by a perspiration sensor is used, the determining unit A detects the perspiration data exceeding a threshold serving as an alert level and makes a notification of prompting water supply, and the determining unit B detects the perspiration data exceeding a threshold serving as a risk level and makes a notification of prompting movement to a cool place to take a rest.

### (Determining Unit 3)

The determining unit preferably includes one or more types of functions selected from a group including functions A, B, and C below.
Function A) a function that converts primary data being measurement data of the measuring unit into secondary data serving as an evaluation index of a dehydration symptom
Function B) a function that detects a sign of a dehydration symptom from a determination reference stored in advance, and the primary data and/or the secondary data
Function C) a function that determines a method of the notification based on the measurement data by the measuring unit

A sign of the dehydration symptom is preferably detected with a determination reference of the B), which is such a determination reference that measurement data to be acquired from a biosensor that acquires one or more types of measurement information selected from a group including an activity amount, a heart rate, a blood pressure, an oxygen intake amount, a respiratory rate, a deep body temperature, a skin temperature, a skin impedance, perspiration amount, and a blood flow rate remains high.

The “remaining high” in which a value of measurement data by one or more types selected from a group including an activity amount, a heart rate, a blood pressure, an oxygen intake amount, a respiratory rate, a deep body temperature, a skin temperature, a skin impedance, a perspiration amount, and a blood flow rate remains in a high state occurs at a substantial initial stage of the dehydration symptom, so that it is possible to avoid a risk of falling into a serious dehydration symptom by detecting a sign of the dehydration symptom using the data remaining high as a determination reference and making a suitable notification.

It is preferable to use in combination a determining unit that uses remaining high as a determination reference and another determining unit that detects a state where the dehydration symptom has progressed.

The determination method in the C) may preferably include one or more types selected from a group including notification timing, a notification method, a notification item, and notification intensity.

### Third Embodiment_Measuring Unit+Informing Unit+Determining Unit+Holding Unit

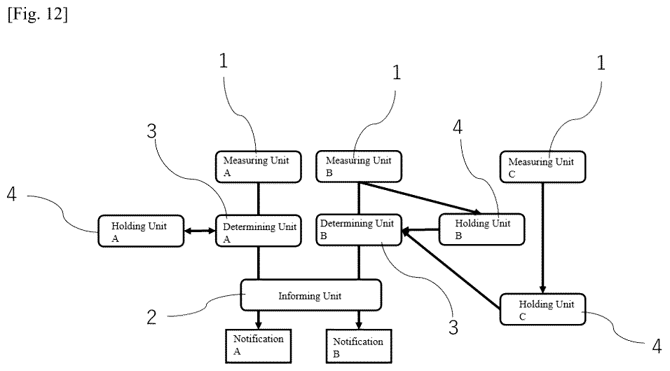

A dehydration notification system in one embodiment of the present invention includes, in addition to the second embodiment, as illustrated in FIGS. 12 to 14, a holding unit 4 that stores measurement data, and derives the determination reference by using measurement data stored in the holding unit.

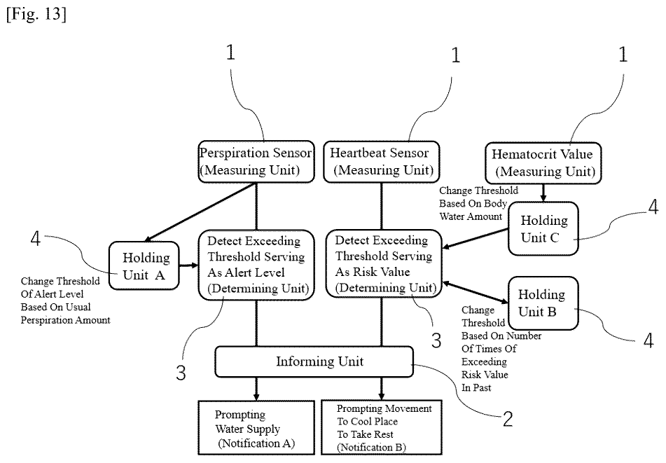

As illustrated in the embodiment in FIG. 13, in addition to the embodiment in which when the perspiration amount exceeding a threshold serving as an alert level is detected using perspiration data measured by a perspiration sensor and a notification of prompting water supply is made, with reference to stored data in a holding unit A that stores perspiration data measured by a perspiration sensor, a threshold of the alert level is changed based on a usual perspiration amount, when the heart rate exceeding a threshold serving as a risk value is detected using heartbeat data measured by a heartbeat sensor and a notification of prompting movement to a cool place to take a rest is made, a threshold can be changed based on stored data in a holding unit B that stores a hematocrit value measured by a body water amount measuring unit, and stored data in a holding unit C that stores the number of times of the heart rate exceeding a threshold serving as a risk value in the past. A threshold serving as a determination reference is changed with reference to the stored data in this manner, so that it is possible to prompt a suitable action.

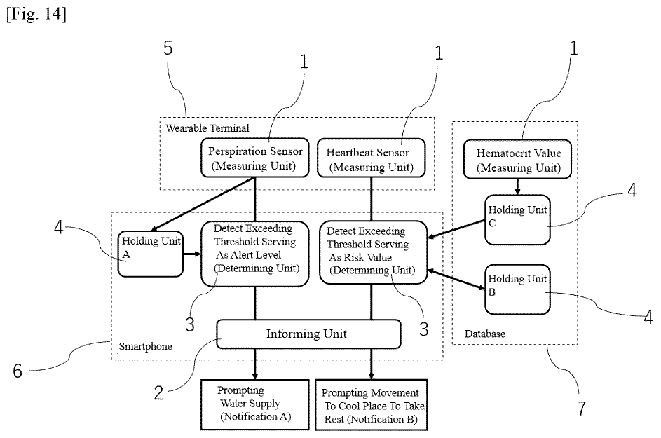

As illustrated in FIG. 14, a hardware configuration including a wearable terminal 5 provided with a perspiration sensor and a heartbeat sensor, a smartphone 6 provided with the holding unit A, the respective determining units, and an informing unit, and a database 7 including the holding unit B and the holding unit C can be constructed.

### Embodiment 4 Measuring Unit+Informing Unit “Two Types of Measurement, One Type of Notification”

A dehydration notification system according to one embodiment of the present invention includes a measuring unit that performs two or more types of measurement selected from a group including water loss amount variation factor measurement, water loss amount measurement, body influence measurement, body water amount measurement, supplied water amount measurement, body feature measurement, and lifestyle measurement, and an informing unit that performs notifications being one or more types of notifications that are derived from two or more types of measurement data selected from a group including water loss amount variation factor measurement data, water loss amount measurement data, body influence measurement data, body water amount measurement data, supplied water amount measurement data, body feature measurement data, and lifestyle measurement data, which are obtained from two or more types of the measurement, and being notifications related to a dehydration symptom.

Making a notification by integrating two or more types of measurement data can more effectively avoid the occurrence of a dehydration symptom, compared with the technique of merely determining the presence or absence of a risk of a dehydration symptom during measurement by a comparison between vital data during measurement and vital data during normal state time.

### REFERENCE SIGNS LIST

1: Measuring unit
2: Informing unit
3: Determining unit
4: Holding unit
5: Wearable terminal
6: Smartphone
7: Database

1. A dehydration notification system comprising:
one or more types of measuring units that perform one or more types of measurement selected from the group consisting of water loss amount variation factor measurement, water loss amount measurement, body influence measurement, body water amount measurement, supplied water amount measurement, body feature measurement, and lifestyle measurement; and
an informing unit that makes notifications being two or more types of notifications that are derived from two or more types of measurement data selected from the group consisting of water loss amount variation factor measurement data, water loss amount measurement data, body influence measurement data, body water amount measurement data, supplied water amount measurement data, body feature measurement data, and lifestyle measurement data, which are obtained from the measurement, and being notifications related to a dehydration symptom.

2. The dehydration notification system according to claim 1, wherein the notification is selected from the group consisting of a notification related to an action for avoiding an occurrence of a dehydration symptom and/or alleviating a symptom of the dehydration symptom, a notification related to a degree of risk that a dehydration symptom occurs, and a notification related to severity of a dehydration symptom.

3. The dehydration notification system according to claim 1, further comprising a determining unit that derives the notification from the measurement data.

4. The dehydration notification system according to claim 3, wherein the determining unit derives one type of a notification from two or more types of measurement data.

5. The dehydration notification system according to claim 3 comprising two or more types of the determining units, wherein at least two types of the determining units respectively derive notifications different from each other from one type of common measurement data.

6. The dehydration notification system according to claim 3, wherein the determining unit includes one or more types of functions selected from the group consisting of A, B, and C;
Function A) a function that converts primary data being measurement data of the measuring unit into secondary data serving as an evaluation index of a dehydration symptom;
Function B) a function that detects a sign of a dehydration symptom from a determination reference stored in advance, and the primary data and/or the secondary data; and
Function C) a function that determines a method of the notification based on the primary data and/or the secondary data.

7. The dehydration notification system according to claim 6, wherein the method determined in the C) includes one or more types selected from the group consisting of notification timing, a notification method, a notification item, and notification intensity.

8. The dehydration notification system according to claim 6, wherein a determination reference of the B) is in that measurement data to be acquired from a biosensor that acquires one or more types of measurement information selected from the group consisting of an activity amount, a heart rate, a blood pressure, an oxygen intake amount, a respiratory rate, a deep body temperature, a skin temperature, a skin impedance, a perspiration amount, and a blood flow rate remains high.

9. The dehydration notification system according to claim 6, further comprising a holding unit that stores the measurement data, wherein the determination reference is derived using the measurement data stored in the holding unit.

10. The dehydration notification system according to claim 1, wherein the measuring unit is any one selected from the group consisting of (A) to (D):
(A) a perspiration sensor including one or more types of sensors that measure an amount of body water to be generated from a skin surface, and one or more types of evaporation promotion mechanisms that prompt evaporation;
(B) a perspiration sensor including two or more types of sensors that measure an amount of body water to be generated from a skin surface;
(C) a perspiration sensor including one or more types of sensors that measure an amount of body water to be generated from a skin surface, a moisture-proof mechanism, and a discharge unit of a liquid; and
(D) a perspiration sensor including: a housing capsule including an opening portion to be attached to a skin surface, the housing capsule including an air suction hole for sucking natural air into an inside of the housing capsule, a mixture chamber that communicates with the opening portion to diffuse sweat on the skin surface, and in which the diffused sweat and the natural air are mixed to become mixed air, and an air discharge hole for discharging the mixed air from the mixture chamber; a first humidity sensor for measuring humidity of the natural air; and a second humidity sensor for measuring humidity of the mixed air.

11. The dehydration notification system according to claim 1, wherein
the measuring unit is a biosensor to be used by being worn on a body, and
the biosensor acquires one or more types of measurement information selected from the group consisting of an activity amount of the body, a heart rate, a pulse wave, a blood pressure, an oxygen intake amount, a respiratory rate, a deep body temperature, a skin temperature, a skin impedance, a perspiration amount, and a blood flow rate.

12. The dehydration notification system according to claim 1, wherein the measuring unit is a unit that measures a content rate of blood hemoglobin and/or a unit that measures an NaCl concentration in sweat.

13. The dehydration notification system according to claim 1, wherein the measuring unit is a unit that measures one or more types selected from the group consisting of blood vasopressin concentration, a urine specific gravity, a urine osmotic pressure, and a urine volume.

14. The dehydration notification system according to claim 1, wherein the measuring unit is a moisture measuring unit that uses a bioelectric impedance analysis method and/or a unit that measures a hematocrit value.

15. The dehydration notification system according to claim 1, wherein the measuring unit acquires eating-and-drinking information including an intake amount and a frequency of food and drink having a diuretic effect and/or exercise information including an exercise habit and an exercise experience.

16. The dehydration notification system according to claim 1, wherein the measuring unit acquires one or more types of information selected from the group consisting of a water intake amount, an intake habit of food and drink having a diuretic effect, an exercise habit, and an exercise experience.

17. The dehydration notification system according to claim 1, wherein the measuring unit acquires supplied water amount information.

18. The dehydration notification system according to claim 1, wherein the measuring unit acquires information including one or more types selected from the group consisting of gender, an age, a BMI, and a body composition.

19. The dehydration notification system according to claim 1, wherein the measuring unit acquires information including one or more types selected from the group consisting of a season, a temperature, a humidity, and a heat index (WBGT).

20. A dehydration notification system comprising:
a measuring unit that performs two or more types of measurement selected from the group consisting of water loss amount variation factor measurement, water loss amount measurement, body influence measurement, body water amount measurement, supplied water amount measurement, body feature measurement, and lifestyle measurement; and
an informing unit that makes notifications being one or more types of notifications that are derived from two or more types of measurement data selected from the group consisting of water loss amount variation factor measurement data, water loss amount measurement data, body influence measurement data, body water amount measurement data, supplied water amount measurement data, body feature measurement data, and lifestyle measurement data, which are obtained from the two or more types of the measurement, and being notifications related to a dehydration symptom.
